(19) United States
(12) Patent Application Publication
(10) Pub. No.: US 2011/0089021 A1
(43) Pub. Date: Apr. 21, 2011

(54) Method For Producing Amides In The Presence Of Superheated Water

(75) Inventors: Matthias Krull, Harxheim (DE); Roman Morschhaeuser, Mainz (DE)

(73) Assignee: CLARIANT FINANCE (BVI) LIMITED, Tortola (VG)

(21) Appl. No.: 12/935,720

(22) PCT Filed: Mar. 18, 2009
(86) PCT No.: PCT/EP2009/001988

§ 371 Date: Dec. 8, 2010

(30) Foreign Application Priority Data
Apr. 4, 2008 (DE) 10 2008 017 219.7

(51) Int. Cl. C07C 51/06

(52) U.S. Cl. 204/157.81

(57) ABSTRACT

The invention relates to a method for producing carboxylic acid amides, according to which at least one carboxylic acid of formula (I)

R³—COON (I)

wherein R³ is hydrogen or an optionally substituted hydrocarbon radical comprising between 1 and 50 carbon atoms, is reacted with at least one amine of formula (II)

HNR¹R² (II)

wherein R¹ and R² are independently hydrogen or an optionally substituted hydrocarbon radical comprising between 1 and 100 C atoms, to form an ammonium salt, and said ammonium salt is reacted in the presence of superheated water, under microwave irradiation, to form a carboxylic acid amide.

The present invention relates to a process for preparing amides under microwave irradiation, wherein the ammonium salt of at least one carboxylic acid and at least one amine is condensed to give the amide in the presence of superheated water.

Carboxamides find various uses as chemical raw materials. For example, carboxamides with low molecular weight have outstanding properties as a solvent, whereas carboxamides bearing at least one relatively long alkyl radical are surface-active. For instance, carboxamides are used, inter alia, as a solvent and as a constituent of washing and cleaning products and in cosmetics. They are additionally used successfully as assistants in metalworking, in the formulation of crop protection products, as antistats for polyolefins and in the delivery and processing of mineral oil. Furthermore, carboxamides are also important raw materials for production of a wide variety of different pharmaceuticals and agrochemicals.

A relatively recent approach to the synthesis of carboxamides is the microwave-supported direct conversion of carboxylic acids and amines to amides. In contrast to conventional thermal processes, this does not require activation of the carboxylic acid by means of, for example, acid chlorides, acid anhydrides, esters or coupling reagents, which makes this process very economically and also ecologically interesting.

Vázquez-Tato, Synlett 1993, 506 discloses the use of microwaves as a heat source for the preparation of amides from carboxylic acids and arylaliphatic amines via the ammonium salts.

Gelens et al., Tetrahedron Letters 2005, 46(21), 3751-3754 discloses a multitude of amides which have been synthesized with the aid of microwave radiation.

Goretzki et. al., Macromol. Rapid Commun. 2004, 25, 513-516 discloses the microwave-supported synthesis of different (meth)acrylamides directly from (meth)acrylic acid and primary amines.

The conversions attained in the microwave-supported syntheses of amides from carboxylic acid and amine described to date are, however, generally still unsatisfactory for commercial applications. Thus, additional isolation and workup steps have to be carried out in order to remove unconverted reactants in particular from the reaction mixture. Since amidations are equilibrium reactions, for the purpose of shifting the equilibrium in the direction of the amide, the content in the reaction mixture of water and especially of water of reaction is kept to a minimum, which is accomplished in batchwise processes, for example, by separating out water with entraining agents during the condensation or by applying reduced pressure. In continuous processes, especially in the case of processes performed under elevated pressure, a removal of the water of reaction is, however, barely possible. Accordingly, Katritzky et al. (Energy & Fuels 4 (1990), 555-561) describe the hydrolysis of tertiary amides to carboxylic acids with partial subsequent decarboxylation for aquathermal processes, and An et al. (J. Org. Chem. (1997), 62, 2505-2511) for microwave-supported processes in superheated water. This involves hydrolyzing various amides and also various nitriles via the state of the amide to carboxylic acids.

A problem in the synthesis of amides from carboxylic acid and amine is often also the relative volatility of the reactants used, which necessitates extensive technical measures for the handling thereof. Moreover, the heat of neutralization which occurs in the course of preparation of the ammonium salts formed as intermediates requires, especially in the case of relatively volatile amines and/or carboxylic acids, intensive cooling and/or long mixing or reaction times. It was therefore an object of the present invention to develop a process with which the conversions in microwave-supported amidations proceeding from carboxylic acid and amine can be increased, and in which the disadvantages of the prior art mentioned are additionally reduced.

It has been found that, surprisingly, the conversion in amidation reactions in which at least one amine and at least one carboxylic acid are converted to an ammonium salt and then to the amide under microwave irradiation can be increased significantly by the presence of superheated water. This was all the more surprising in that such condensation reactions which proceed with elimination of water are subject to the law of mass action, and the increase in the concentration of one of the reaction products accordingly typically shifts the equilibrium in the direction of the reactants. In addition, it is possible in this process to use aqueous solutions, especially of low-boiling reactants, such that these need not be handled under pressure or in cooled form. Furthermore, in the course of preparation of the ammonium salt, the presence of water results in improved heat removal.

The invention provides a process for preparing carboxamides by reacting at least one carboxylic acid of the formula I

R³—COON (I)

in which R³ is hydrogen or an optionally substituted hydrocarbon radical having 1 to 50 carbon atoms
with at least one amine of the formula II

HNR¹R² (II)

in which R¹ and R² are each independently hydrogen or an optionally substituted hydrocarbon radical having 1 to 100 carbon atoms to give an ammonium salt, and this ammonium salt is converted to the carboxamide in the presence of superheated water under microwave irradiation.

The invention further provides a process for preparing carboxamides by reacting at least one carboxylic acid of the formula I

R³—COON (I)

in which R³ is hydrogen or an optionally substituted hydrocarbon radical having 1 to 50 carbon atoms
with at least one amine of the formula II

HNR¹R² (II)

in which R¹ and R² are each independently hydrogen or an optionally substituted hydrocarbon radical having 1 to 100 carbon atoms
in the presence of water to give an ammonium salt, and the water-containing ammonium salt thus prepared is converted to the carboxamide at temperatures above 100° C. under microwave irradiation.

The invention further provides a process for increasing the conversion of microwave-supported amidation reactions, in which water is added before microwave irradiation to an ammonium salt of at least one carboxylic acid of the formula I

R³—COON (I)

in which R³ is hydrogen or an optionally substituted hydrocarbon radical having 1 to 50 carbon atoms
and at least one amine of the formula II

HNR¹R² (II)

in which R¹ and R² are each independently hydrogen or an optionally substituted hydrocarbon radical having 1 to 100 carbon atoms.

Suitable carboxylic acids of the formula I are generally compounds which possess at least one carboxyl group. Thus, the process according to the invention is likewise suitable for conversion of carboxylic acids having, for example, two, three, four or more carboxyl groups. The carboxylic acids may be of natural or synthetic origin. As well as formic acid, particular preference is given to those carboxylic acids which bear a hydrocarbon radical R³ having 1 to 30 carbon atoms and especially having 2 to 24 carbon atoms. The hydrocarbon radical is preferably aliphatic, cycloaliphatic, aromatic or araliphatic. The hydrocarbon radical may bear one or more, for example two, three, four or more, further substituents, for example hydroxyl, hydroxyalkyl, alkoxy, for example methoxy, poly(alkoxy), poly(alkoxy)alkyl, carboxyl, ester, amid, cyano, nitrile, nitro, sulfo and/or C₅-C₂₀-aryl groups, for example phenyl groups, with the proviso that the substituents are stable under the reaction conditions and do not enter into any side reactions, for example elimination reactions. The C₅-C₂₀-aryl groups may themselves in turn bear substituents, for example halogen atoms, halogenated alkyl radicals, C₁-C₂₀-alkyl, C₂-C₂₀-alkenyl, C₁-C₅-alkoxy, for example methoxy, ester, amide, cyano, nitrile and/or nitro groups. The hydrocarbon radical R³ may also contain heteroatoms, for example oxygen, nitrogen, phosphorus and/or sulfur, but preferably not more than one heteroatom per 3 carbon atoms. The reaction of polycarboxylic acids with ammonia or primary amines by the process according to the invention can also form imides.

Preferred carboxylic acids bear aliphatic hydrocarbon radicals. Particular preference is given to aliphatic hydrocarbon radicals having 2 to 24 and especially having 3 to 20 carbon atoms. These aliphatic hydrocarbon radicals may be linear, branched or cyclic. The carboxyl group may be bonded to a primary, secondary or tertiary carbon atoms. The hydrocarbon radicals may be saturated or unsaturated. Unsaturated hydrocarbon radicals contain one or more and preferably one, two or three C═C double bonds. For instance, the process according to the invention has been found to be particularly useful for preparation of amides and especially of polyunsaturated fatty acids, since the double bonds of the unsaturated fatty acids are not attacked under the reaction conditions of the process according to the invention. In a preferred embodiment, the aliphatic hydrocarbon radical is an unsubstituted alkyl or alkenyl radical. In a further preferred embodiment, the aliphatic hydrocarbon radical bears one or more, for example two, three or more, of the abovementioned substituents.

Preferred cycloaliphatic hydrocarbon radicals are aliphatic hydrocarbon radicals having 2 to 24 and especially having 3 to 20 carbon atoms, and optionally one or more heteroatoms, for example nitrogen, oxygen or sulfur, which possess at least one ring with four, five, six, seven, eight or more ring atoms. The carboxyl group is bonded to one of the rings.

Suitable aliphatic or cycloaliphatic carboxylic acids are, for example, formic acid, acetic acid, propionic acid, butyric acid, isobutyric acid, pentanoic acid, isopentanoic acid, pivalic acid, hexanoic acid, cyclohexanoic acid, heptanoic acid, octanoic acid, nonanoic acid, isononanoic acid, neononanoic acid, decanoic acid, isodecanoic acid, neodecanoic acid, undecanoic acid, neoundecanoic acid, dodecanoic acid, tridecanoic acid, tetradecanoic acid, 12-methyltridecanoic acid, pentadecanoic acid, 13-methyltetradecanoic acid, 12-methyltetradecanoic acid, hexadecanoic acid, 14-methylpentadecanoic acid, heptadecanoic acid, 15-methylhexadecanoic acid, 14-methylhexadecanoic acid, octadecanoic, isooctadecanoic acid, eicosanoic acid, docosanoic acid and tetracosanoic acid, and also myristoleic acid, palmitoleic acid, hexadecadienoic acid, delta-9-cis-heptadecenoic acid, oleic acid, petroselic acid, vaccenic acid, linoleic acid, linolenic acid, gadoleic acid, gondoic acid, eicosadienoic acid, arachidonic acid, cetoleic acid, erucic acid, docosadienoic acid and tetracosenoic acid, and also malonic acid, succinic acid, butanetetracarboxylic acid, dodecenylsuccinic acid and octadecenylsuccinic acid. Additionally suitable are fatty acid mixtures obtainable from natural fats and oils, for example cottonseed oil, coconut oil, groundnut oil, safflower oil, corn oil, palm kernel oil, rapeseed oil, castor oil, olive oil, mustardseed oil, soya oil, sunflower oil, and also tallow oil, bone oil and fish oil. Likewise suitable as fatty acids or fatty acid mixtures for the process according to the invention are tall oil fatty acid, and also resin acids and naphthenic acids.

In a preferred embodiment, the process according to the invention is particularly suitable for preparation of amides of ethylenically unsaturated carboxylic acids, i.e. of carboxylic acids which possess a C═C double bond conjugated to the carboxyl group. Examples of preferred ethylenically unsaturated carboxylic acids are acrylic acid, methacrylic acid, crotonic acid, 2,2-dimethylacrylic acid, senecioic acid, maleic acid, fumaric acid, itaconic acid, cinnamic acid and methoxycinnamic acid.

In a further preferred embodiment, the process according to the invention is particularly suitable for preparation of amides of hydroxycarboxylic acids, i.e. of carboxylic acids which bear at least one hydroxyl group on the aliphatic hydrocarbon radical R³. The hydroxyl group may be bonded to a primary, secondary or tertiary carbon atom. The process is particularly advantageous for the amidation of hydroxycarboxylic acids which contain one hydroxyl group bonded to such a secondary carbon atom, and especially for the amidation of those hydroxycarboxylic acids in which the hydroxyl group is in the α or β position to the carboxyl group. The carboxyl and hydroxyl groups may be bonded to the same or different carbon atoms in R³. The process according to the invention is likewise suitable for amidation of hydroxypolycarboxylic acids having, for example, two, three, four or more carboxyl groups. In addition, the process according to the invention is suitable for amidation of polyhydroxycarboxylic acids having, for example, two, three, four or more hydroxyl groups, though the hydroxycarboxylic acids may bear only one hydroxyl group per carbon atom of the aliphatic hydrocarbon radical R³. Particular preference is given to hydroxycarboxylic acids which bear an aliphatic hydrocarbon radical R³ having 1 to 30 carbon atoms and especially having 2 to 24 carbon atoms, for example having 3 to 20 carbon atoms. In the conversion of the hydroxycarboxylic acids by the process according to the invention, there is neither aminolysis nor elimination of the hydroxyl group.

Suitable aliphatic hydroxycarboxylic acids are, for example, hydroxyacetic acid, 2-hydroxypropionic acid, 3-hydroxypropionic acid, 2-hydroxybutyric acid, 3-hydroxybutyric acid, 4-hydroxybutyric acid, 2-hydroxy-2-methylpropionic acid, 4-hydroxypentanoic acid, 5-hydroxypentanoic acid, 2,2-dimethyl-3-hydroxypropionic acid, 5-hydroxyhexanoic acid, 2-hydroxyoctanoic acid, 2-hydroxytetradecanoic acid, 15-hydroxypentadecanoic acid, 16-hydroxyhexadecanoic acid, 12-hydroxystearic acid and α-hydroxyphenylacetic acid, 4-hydroxymandelic acid, 2-hydroxy-2-phenylpropionic acid and 3-hydroxy-3-phenylpropionic acid. It is also possible to convert hydroxypolycarboxylic acids, for example hydroxysuccinic acid, citric acid and isocitric acid, polyhydroxycarboxylic acids, for example gluconic acid, and polyhydroxypolycarboxylic acids, for example tartaric acid, to the corresponding amides with increased conversions by means of the process according to the invention.

Additionally preferred carboxylic acids bear aromatic hydrocarbon radicals R³. Such aromatic carboxylic acids are understood to mean compounds which bear at least one carboxyl group bonded to an aromatic system (aryl radical). Aromatic systems are understood to mean cyclic, through-conjugated systems with (4n+2) Tr electrons, in which n is a natural whole number and is preferably 1, 2, 3, 4 or 5. The aromatic system may be mono- or polycyclic, for example di- or tricyclic. The aromatic system is preferably formed from carbon atoms. In a further preferred embodiment, it contains, as well as carbon atoms, one or more heteroatoms, for example nitrogen, oxygen and/or sulfur. Examples of such aromatic systems are benzene, naphthalene, phenanthrene, furan and pyridine. The aromatic system may, as well as the carboxyl group, bear one or more, for example one, two, three or more, identical or different further substituents. Suitable further substituents are, for example, alkyl, alkenyl and halogenated alkyl radicals, hydroxyl, hydroxyalkyl, alkoxy, halogen, cyano, nitrile, nitro and/or sulfo groups. These may be bonded to any position in the aromatic system. However, the aryl radical bears at most as many substituents as it has valences.

In a specific embodiment, the aryl radical bears further carboxyl groups. Thus, the process according to the invention is likewise suitable for conversion of aromatic carboxylic acids having, for example, two or more carboxyl groups. The reaction of polycarboxylic acids with ammonia or primary amines by the process according to the invention can also form imides, especially when the carboxyl groups are in the ortho position on an aromatic system.

The process according to the invention is particularly suitable for amidation of alkylarylcarboxylic acids, for example alkylphenylcarboxylic acids. These are aromatic carboxylic acids in which the aryl radical bearing the carboxyl group additionally bears at least one alkyl or alkylene radical. The process is particularly advantageous in the amidation of alkylbenzoic acids which bear at least one alkyl radical having 1 to 20 carbon atoms and especially 1 to 12 carbon atoms, for example 1 to 4 carbon atoms.

The process according to the invention is additionally particularly suitable for amidation of aromatic carboxylic acids whose aryl radical bears one or more, for example two or three, hydroxyl groups and/or hydroxyalkyl groups. In the amidation with at least equimolar amounts of amine of the formula (II), selective amidation of the carboxyl group takes place; no esters and/or polyesters are formed.

Suitable aromatic carboxylic acids are, for example, benzoic acid, phthalic acid, isophthalic acid, the different isomers of naphthalenecarboxylic acid, pyridine-carboxylic acid and naphthalenedicarboxylic acid, and also trimellitic acid, trimesic acid, pyromellitic acid and mellitic acid, the different isomers of methoxybenzoic acid, hydroxybenzoic acid, hydroxymethylbenzoic acid, hydroxymethoxybenzoic acid, hydroxydimethoxybenzoic acid, hydroxyisophthalic acid, hydroxynaphthalenecarboxylic acid, hydoxypyridinecarboxylic acid and hydroxymethylpyridinecarboxylic acid, hydroxyquinolinecarboxylic acid, and also o-toluic acid, m-toluic acid, p-toluic acid, o-ethylbenzoic acid, m-ethylbenzoic acid, p-ethylbenzoic acid, o-propylbenzoic acid, m-propylbenzoic acid, p-propylbenzoic acid and 3,4-dimethylbenzoic acid.

Further preferred carboxylic acids bear araliphatic hydrocarbon radicals R³. Such araliphatic carboxylic acids bear at least one carboxyl group bonded via an alkylene or alkylenyl radical to an aromatic system. The alkylene or alkenylene radical preferably has 1 to 10 carbon atoms and especially 2 to 5 carbon atoms. It may be linear or branched, preferably linear. Preferred alkylenylene radicals possess one or more, for example one, two or three, double bonds. An aromatic system is understood to mean the aromatic systems already defined above, to which the at least one alkyl radical bearing a carboxyl group is bonded. The aromatic systems may themselves in turn bear substituents, for example halogen atoms, halogenated alkyl radicals, C₁-C₂₀-alkyl, C₂-C₂₀-alkenyl, C₁-C₅-alkoxy, for example methoxy, hydroxyl, hydroxyalkyl, ester, amide, cyano, nitrile and/or nitro groups. Examples of preferred araliphatic carboxylic acids are phenylacetic acid, (2-bromophenyl)acetic acid, 3-(ethoxyphenyl)acetic acid, 4-(methoxyphenyl)acetic acid, (dimethoxyphenyl)acetic acid, 2-phenylpropionic acid, 3-phenylpropionic acid, 3-(4-hydroxyphenyl)propionic acid, 4-hydroxyphenoxyacetic acid, cinnamic acid and mixtures thereof.

Mixtures of different carboxylic acids are also suitable for use in the process according to the invention.

The process according to the invention is preferentially suitable for preparation of secondary amides, i.e. for conversion of amines in which R¹ is a hydrocarbon radical having 1 to 100 carbon atoms and R² is hydrogen.

The process according to the invention is additionally preferentially suitable for preparation of tertiary amines, i.e. for reaction of carboxylic acids with amines, in which both R¹ and R² radicals are independently a hydrocarbon radical having 1 to 100 carbon atoms. The R¹ and R² radicals may be the same or different. In a particularly preferred embodiment, R¹ and R² are the same.

In a first preferred embodiment, R¹ and/or R² are each independently an aliphatic radical. This radical has preferably 1 to 24, more preferably 2 to 18 and especially 3 to 6 carbon atoms. The aliphatic radical may be linear, branched or cyclic. It may additionally be saturated or unsaturated. The aliphatic radical is preferably saturated. The aliphatic radical may bear substituents, for example hydroxyl, C₁-C₅-alkoxy, cyano, nitrile, nitro and/or C₅-C₂₀-aryl groups, for example phenyl radicals. The C₅-C₂₀-aryl radicals may themselves optionally be substituted by halogen atoms, halogenated alkyl radicals, C₁-C₂₀-alkyl, C₂-C₂₀-alkenyl, hydroxyl, C₁-C₅-alkoxy, for example methoxy, amide, cyano, nitrile and/or nitro groups. In a particularly preferred embodiment, R¹ and/or R² are each independently hydrogen, a C₁-C₆-alkyl, C₂-C₆-alkenyl or C₃-C₆-cycloalkyl radical, and especially an alkyl radical having 1, 2 or 3 carbon atoms. These radicals may bear up to three substituents. Particularly preferred aliphatic R¹ and/or R² radicals are hydrogen, methyl, ethyl, hydroxyethyl, n-propyl, isopropyl, hydroxypropyl, n-butyl, isobutyl and tert-butyl, hydroxybutyl, n-hexyl, cyclohexyl, n-octyl, n-decyl, n-dodecyl, tridecyl, isotridecyl, tetradecyl, hexadecyl, octadecyl and methylphenyl.

In a further preferred embodiment, R¹ and R² together with the nitrogen atom to which they are bonded form a ring. This ring preferably has 4 or more, for example 4, 5, 6 or more, ring members. Preferred further ring members are carbon, nitrogen, oxygen and sulfur atoms. The rings may themselves in turn bear substituents, for example alkyl radicals. Suitable ring structures are, for example, morpholinyl, pyrrolidinyl, piperidinyl, imidazolyl and azepanyl radicals.

In a further preferred embodiment, R¹ and/or R² are each independently an optionally substituted C₆-C₁₂-aryl group or an optionally substituted heteroaromatic group having 5 to 12 ring members.

In a further preferred embodiment, R¹ and/or R² are each independently an alkyl radical interrupted by heteroatoms. Particularly preferred heteroatoms are oxygen and nitrogen.

For instance, R¹ and/or R² are preferably each independently radicals of the formula III

—(R⁴—O)ₙ—R⁵ (III)

in which
R⁴ is an alkylene group having 2 to 6 carbon atoms and preferably having 2 to 4 carbon atoms, for example ethylene, propylene, butylene or mixtures thereof,
R⁵ is hydrogen, a hydrocarbon radical having 1 to 24 carbon atoms or a group of the formula —NR¹⁰R¹¹,
n is from 2 to 50, preferably from 3 to 25 and especially from 4 to 10, and
R¹⁰, R¹¹ are each independently hydrogen, an aliphatic radical having 1 to 24 carbon atoms and preferably 2 to 18 carbon atoms, an aryl group or heteroaryl group having 5 to 12 ring members, a poly(oxyalkylene) group having 1 to 50 poly(oxyalkylene) units, where the polyoxyalkylene units derived from alkylene oxide units having 2 to 6 carbon atoms, or R¹⁰ and R¹¹ together with the nitrogen atom to which they are bonded form a ring having 4, 5, 6 or more ring members.

Additionally preferably, R¹ and/or R² are each independently radicals of the formula IV

—[R⁶—N(R⁷)]ₘ—(R⁷) (IV)

in which
R⁶ is an alkylene group having 2 to 6 carbon atoms and preferably having 2 to 4 carbon atoms, for example ethylene, propylene or mixtures thereof,
each R⁷ is independently hydrogen, an alkyl or hydroxyalkyl radical having up to 24 carbon atoms, for example 2 to 20 carbon atoms, a polyoxyalkylene radical —(R⁴—O)ₚ—R⁵, or a polyiminoalkylene radical —[R⁶—N(R⁷)]_{q}—(R⁷), where R⁴, R⁵, R⁶ and R⁷ are each as defined above and q and p are each independently 1 to 50, and
m is from 1 to 20 and preferably 2 to 10, for example three, four, five or six. The radicals of the formula IV contain preferably 1 to 50 and especially 2 to 20 nitrogen atoms.

According to the stoichiometric ratio between aromatic carboxylic acid (I) and polyamine (IV), one or more amino groups which each bear at least one hydrogen atom are converted to the carboxamide. In the reaction of polycarboxylic acids with polyamines of the formula IV, the primary amino groups in particular can also be converted to imides.

For the inventive preparation of primary amides, instead of ammonia, preference is given to using nitrogen compounds which eliminate ammonia gas when heated. Examples of such nitrogen compounds are urea and formamide.

Examples of suitable amines are ammonia, methylamine, ethylamine, ethanolamine, propylamine, propanolamine, butylamine, hexylamine, cyclohexylamine, octylamine, decylamine, dodecylamine, tetradecylamine, hexadecylamine, octadecylamine, dimethylamine, diethylamine, diethanolamine, ethylmethylamine, di-n-propylamine, di-isopropylamine, dicyclohexylamine, didecylamine, didodecylamine, ditetradecylamine, dihexadecylamine, dioctadecylamine, benzylamine, phenylethylamine, ethylenediamine, diethylenetriamine, triethylenetetramine, tetraethylenepentamine, N,N-dimethylethylenediamine, N,N-diethylaminopropylamine, N,N-dimethylaminopropylamine, N,N-(2′-hydroxy-ethyl)-1,3-propanediamine and 1-(3-aminopropyl)pyrrolidine, and mixtures thereof. Among these, particular preference is given to dimethylamine, diethylamine, diethanolamine, di-n-propylamine, diisopropylamine, ethylmethylamine and N,N-dimethylaminopropylamine.

The process according to the invention is particularly suitable for preparation of amides from saturated C₁-C₅-carboxylic acids and primary alkyl- and/or arylamines, from saturated C₁-C₅-carboxylic acids and secondary alkyl- and/or arylamines, from saturated C₁-C₅-carboxylic acids and amines bearing hydroxyl groups, from saturated C₁-C₅-carboxylic acids and polyetheramines, from saturated C₁-C₅-carboxylic acids and polyamines, from aliphatic hydroxycarboxylic acids and primary alkyl- and/or arylamines, from aliphatic hydroxycarboxylic acids and secondary alkyl- and/or arylamines, from aliphatic hydroxycarboxylic acids and polyamines, from C₆-C₅₀-alkyl- and/or -alkenylcarboxylic acids and polyetheramines, from C₆-C₅₀-alkyl- and/or -alkenylcarboxylic acids and polyamines, from C₆-C₅₀-alkyl- and/or -alkenylcarboxylic acids and primary alkyl- and/or arylamines, from C₆-C₅₀-alkyl- and/or -alkenylcarboxylic acids and secondary alkyl- and/or arylamines, from C₆-C₅₀-alkyl- and/or -alkenylcarboxylic acids and amines which bear hydroxyl groups, from C₃-C₅-alkenylcarboxylic acids and primary alkyl- and/or arylamines, from C₃-C₅-alkenylcarboxylic acids and secondary alkyl- and/or arylamines, from C₃-C₅-alkenylcarboxylic acids and amines which bear hydroxyl groups, from C₃-C₅-alkenylcarboxylic acids and polyetheramines, from C₃-C₅-alkenylcarboxylic acids and polyamines, from arylcarboxylic acids which optionally bear hydroxyl groups and primary alkyl- and/or arylamines, arylcarboxylic acids which optionally bear hydroxyl groups and secondary alkyl- and/or arylamines, from arylcarboxylic acids which optionally bear hydroxyl groups and amines which bear hydroxyl groups, from arylcarboxylic acids optionally bearing hydroxyl groups and polyetheramines, and from arylcarboxylic acids which optionally bear hydroxyl groups and polyamines.

The process is especially suitable for preparation of N,N-dimethylformamide, N-octylformamide, N-methylacetamide, N,N-dimethylacetamide, N-ethylacetamide, N,N-diethylacetamide, N,N-dipropylacetamide, N,N-dimethylpropionamide, N,N-dimethylbutyramide, N,N-dimethyl(phenyl)acetamide, N,N-dimethyllactamide, N,N-dimethylacrylamide, N,N-dimethylacrylamide, N,N-diethylmethacrylamide, N,N-diethylacrylamide, N-2-ethylhexylacrylamide, N-2-ethylhexylmethacrylamide, N-methylcocoamide, N,N-dimethylcocoamide, N-methylglycolamide, N-ethylmandelamide, N,N-dimethylglycolamide, N,N-dimethyllactamide, N,N-dimethylricinoleamide, octanoic diethanolamide, lauric monoethanolamide, lauric diethanolamide, tall oil fatty acid diethanolamide, tall oil fatty acid monoethanolamide, N,N-dimethylbenzamide, N,N-diethylbenzamide, nicotinamide, N,N-dimethylnicotinamide, N,N-diethyltoluamide and N,N′-di(acetic acid)ethylened iamide.

In the process according to the invention, carboxylic acid and amine can generally be reacted with one another in any desired ratios. The reaction is preferably effected with molar ratios between carboxylic acid and amine of 10:1 to 1:100, preferably of 2:1 to 1:10, especially of 1.2:1 to 1:3, based in each case on the equivalents of carboxyl and amino groups. In a specific embodiment, carboxylic acid and amine are used in equimolar amounts. In many cases, it has been found to be advantageous to work with an excess of amine, i.e. molar ratios of amine to carboxylic acid of at least 1.01:1.00 and especially between 1.02:1.00 and 5.0:1.0, for example between 2.5:1.0 and 1.1:1.0. This process is particularly advantageous when the amine used is relatively volatile or water-soluble. Relatively volatile means here that the amine has a boiling point at standard pressure of preferably below 250° C., for example below 150° C., and can thus be removed from the amide, optionally together with the water. This can be done, for example, by means of phase separation, extraction or distillation.

In the case that R¹ and/or R² is a hydrocarbon radical substituted by one or more hydroxyl groups, the reaction between carboxylic acid (I) and amine (II) is effected with molar ratios of 1:1 to 1:100, preferably of 1:1.001 to 1:10 and especially of 1:1.01 to 1:5, for example of 1:1.1 to 1:2, based in each case on the molar equivalents of carboxyl groups and amino groups in the reaction mixture.

In the case that the carboxylic acid (I) bears one or more hydroxyl groups, the reaction between carboxylic acid (I) and amine (II) is effected with molar ratios of 1:100 to 1:1, preferably of 1:10 to 1:1.001 and especially of 1:5 to 1:1.01, for example of 1:2 to 1:1.1, based in each case on the molar equivalents of carboxyl groups and amino groups in the reaction mixture.

In the case that R¹ and/or R² is a hydrocarbon radical substituted by one or more hydroxyl groups, and that the carboxylic acid bears one or more hydroxyl groups, the reaction between carboxylic acid (I) and amine (II) is effected in equimolar amounts based on the molar equivalents of carboxyl groups and amino groups in the reaction mixture.

The reaction of amine and carboxylic acid to give the ammonium salt can be performed continuously, batchwise or else in semibatchwise processes. For instance, the ammonium salt can be prepared directly in the reaction vessel (irradiation vessel) intended for the microwave irradiation. It can also be carried out in an upstream (semi)batchwise process, for example in a separate stirred vessel. The ammonium salt is preferably obtained in situ and not isolated. For instance, it has been found to be useful especially for processes on the industrial scale to undertake the reaction of amine and carboxylic acid in the presence of water to give the ammonium salt in a mixing zone, out of which the water-containing ammonium salt, optionally after intermediate cooling, is conveyed into the irradiation vessel. The water may be supplied to the mixing zone as a separate stream or preferably as a solvent or dispersant for amine and/or carboxylic acid. Additionally preferably, the reactants are supplied to the process according to the invention in liquid form. To this end, it is possible to use relatively high-melting and/or relatively high-viscosity reactants, for example in the molten state and/or admixed with water and/or further solvent, for example in the form of a solution, dispersion or emulsion. A catalyst can, if used, be added to one of the reactants or else to the reactant mixture before entry into the irradiation vessel. It is also possible to convert solid, pulverulent and heterogeneous systems by the process according to the invention, in which case merely appropriate technical devices for conveying the reaction mixture are required.

According to the invention, the presence of water is understood to mean that water is added to the ammonium salt formed from carboxylic acid and amine before the irradiation with microwaves, and hence the microwave-supported conversion to the amide takes place in the presence of water. Consequently, the reaction product contains an amount of water exceeding the water of reaction released in the amide formation. Preference is given to adding 0.1 to 5000% by weight, more preferably 1 to 1000% by weight and especially 5 to 100% by weight, for example 10 to 50% by weight, of water to the reaction mixture, based on the total amount of carboxylic acid and amine. In a particularly preferred embodiment, at least one of the carboxylic acid and/or amine reactants is used as an aqueous solution to form the ammonium salt. For example, it has been found to be useful to use especially amines which boil below room temperature, for example ammonia, methylamine, dimethylamine or ethylamine, as, for example, 40-70% aqueous solutions to prepare the ammonium salt. The aqueous dilution of the ammonium salt is subsequently, optionally after further addition of water, exposed to microwave radiation.

According to the invention, superheated water is obtained by performing the microwave irradiation under conditions under which water is heated to temperatures above 100° C. under pressure. The amidation is preferably performed in the presence of water at temperatures above 150° C., more preferably between 180 and 500° C. and especially between 200 and 400° C., for example between 220 and 350° C. These temperatures relate to the maximum temperatures obtained during the microwave irradiation. The pressure is preferably set to a sufficiently high level that the reaction mixture is in the liquid state and does not boil. Preference is given to working at pressures above 1 bar, preferably at pressures between 3 and 300 bar, more preferably between 5 and 200 bar and especially between 10 and 100 bar, for example between 15 and 50 bar.

To accelerate or to complete the reaction, it has been found to be useful in many cases to work in the presence of dehydrating catalysts. Dehydrating catalysts are understood to mean assistants which accelerate the condensation of amine and carboxylic acid. Preference is given to working in the presence of an acidic inorganic, organometallic or organic catalyst, or mixtures of two or more of these catalysts. In a particularly preferred embodiment, no catalyst is employed.

A preferred embodiment works in the presence of additional organic solvents, in order, for example, to lower the viscosity of the reaction medium and/or to fluidize the reaction mixture if it is heterogeneous. For this purpose, it is possible in principle to use all solvents which are inert under the reaction conditions employed and do not react with the reactants or the products formed. When working in the presence of additional solvents, the proportion thereof in the reaction mixture is preferably between 1 and 90% by weight, especially between 5 and 75% by weight and particularly between 10 and 60% by weight, for example between 20 and 50% by weight. Particular preference is given to performing the reaction in the absence of additional solvents.

After the microwave irradiation, the reaction mixture in many cases can be sent directly to a further use. In order to obtain anhydrous products, the water can be removed from the crude product by customary separating processes, for example phase separation, distillation, freeze-drying or absorption. At the same time, it is also possible to additionally remove reactants used in excess and any unconverted residual amounts of the reactants. For specific requirements, the crude products can be purified further by customary purifying processes, for example distillation, recrystallization, filtration or chromatographic processes.

The microwave irradiation is typically performed in instruments which possess a reaction chamber (irradiation vessel) of a substantially microwave-transparent material, into which microwave irradiation generated in a microwave generator is injected. Microwave generators, for example the magnetron, the klystron and the gyrotron, are known to those skilled in the art.

The irradiation vessels used to perform the process according to the invention are preferably manufactured from substantially microwave-transparent, high-melting material or comprise at least parts, for example windows, made of these materials. Particular preference is given to using nonmetallic irradiation vessels. Substantially microwave-transparent materials are understood here to mean those which absorb a minimum amount of microwave energy and convert it to heat. A measure often employed for the ability of a substance to absorb microwave energy and convert it to heat is the dielectric loss factor tan δ=ε″/ε′. The dielectric loss factor tan δ is defined as the ratio of dielectric loss ε″ and dielectric constant ε′. Examples of tan δ values of different materials are reproduced, for example, in D. Bogdal, Microwave-assisted Organic Synthesis, Elsevier 2005. For irradiation vessels suitable in accordance with the invention, materials with tan δ values measured at 2.45 GHz and 25° C. of less than 0.01, particularly less than 0.005 and especially less than 0.001 are preferred. Useful preferred microwave-transparent and thermally stable materials are primarily mineral-based materials, for example quartz, aluminum oxide, zirconium oxide and the like. Also suitable as vessel materials are thermally stable plastics, such as especially fluoropolymers, for example Teflon, and industrial plastics such as polypropylene, or polyaryl ether ketones, for example glass fiber reinforced polyetheretherketone (PEEK). In order to withstand the temperature conditions during the reaction, especially minerals, such as quartz or aluminum oxide, coated with these plastics have been found to be useful as reactor materials.

Microwaves refer to electromagnetic rays with a wavelength between about 1 cm and 1 m and frequencies between about 300 MHz and 30 GHz. This frequency range is suitable in principle for the process according to the invention. Preference is given to using, for the process according to the invention, microwave radiation with frequencies approved for industrial, scientific and medical applications, for example with frequencies of 915 MHz, 2.45 GHz, 5.8 GHz or 27.12 GHz. The microwave irradiation of the ammonium salt can be effected either in microwave applicators which work in monomode or quasi-monomode, or in those which work in multimode. Corresponding instruments are known to those skilled in the art.

The microwave power to be injected into the irradiation vessel for the performance of the process according to the invention is especially dependent on the target reaction temperature, the geometry of the reaction chamber and hence the reaction volume. It is typically between 100 W and several hundreds of kW and especially between 200 W and 100 kW, for example between 500 W and 70 kW. It can be applied at one or more points in the irradiation vessel. It can be obtained by means of one or more microwave generators.

The duration of the microwave irradiation depends on various factors, such as the reaction volume, the geometry of the irradiation vessel, the desired residence time of the reaction mixture at reaction temperature, and the desired degree of conversion. Typically, the microwave irradiation is undertaken over a period of less than 30 minutes, preferably between 0.01 second and 15 minutes, more preferably between 0.1 second and 10 minutes, and especially between one second and 5 minutes, for example between 5 seconds and 2 minutes. The intensity (power) of the microwave radiation is adjusted such that the reaction mixture attains the target reaction temperature within a minimum time. In a further preferred embodiment of the process according to the invention, it has been found to be useful to heat the ammonium salt even before commencement of the microwave irradiation, for which one possible means is to utilize the heat of reaction released in the formation of the ammonium salt. It has been found to be particularly useful to heat the ammonium salt to temperatures between about 40 and about 120° C., but preferably to temperatures below the boiling point of the system. To maintain the target reaction temperature, the reaction mixture can be irradiated further with reduced and/or pulsed power, or kept at temperature by some other means. In a preferred embodiment, the reaction product is cooled directly after the microwave irradiation has ended very rapidly to temperatures below 120° C., preferably below 100° C. and especially below 50° C.

The microwave irradiation can be performed batchwise in a batch process, or preferably continuously, for example in a flow tube. It can additionally be performed in semibatchwise processes, for example continuous stirred reactors or cascade reactors. In a preferred embodiment, the reaction is performed in a closed, pressure-resistant and chemically inert vessel, in which case the water and in some cases the reactants lead to a pressure buildup. After the reaction has ended, the elevated pressure can be used, by decompression, to volatilize and remove water and any excess reactants and/or cool the reaction product. In a further embodiment, the water is removed after the cooling and/or decompression by customary processes, for example phase separation, distillation and/or absorption. In a particularly preferred embodiment, the reaction mixture, after the microwave irradiation has ended or after leaving the irradiation vessel, is freed as rapidly as possible from the excess amine and water in order to avoid hydrolysis of the amide. This can be done, for example, by customary separating processes, such as phase separation, distillation or absorption. It has often also been found to be successful here to neutralize the amine or to admix it with excess acid. This preferably establishes pH values below 7, for example between 1 and 6.5, and especially between 3 and 6.

In a preferred embodiment, the process according to the invention is performed in a batchwise microwave reactor in which a particular amount of the aqueous ammonium salt is charged into an irradiation vessel, irradiated with microwaves and then worked up. The microwave irradiation is preferably undertaken in a pressure-resistant stirred vessel. The microwaves can be injected into the reaction vessel, if the reaction vessel is manufactured from a microwave-transparent material or possesses microwave-transparent windows, through the vessel wall. However, the microwaves can also be injected into the reaction vessel via antennas, probes or hollow conductor systems. For the irradiation of relatively large reaction volumes, the microwave here is preferably operated in multimode. The batchwise embodiment of the process according to the invention allows, through variation of the microwave power, rapid and also slow heating rates, and especially the holding of the temperature over prolonged periods, for example several hours. In a preferred embodiment, the aqueous reaction mixture is initially charged in the irradiation vessel before commencement of the microwave irradiation. It preferably has temperatures below 100° C., for example between 10 and 50° C. In a further preferred embodiment, the reactants and water or parts thereof are supplied to the irradiation vessel only during the irradiation with microwaves. In a further preferred embodiment, the batchwise microwave reactor is operated with continuous supply of reactants and simultaneous discharge of reaction mixture in the form of a semibatchwise or cascade reactor.

In a particularly preferred embodiment, the process according to the invention is performed in a continuous microwave reactor. To this end, the reaction mixture is conducted continuously through a pressure-resistant reaction tube which is inert to the reactants, is very substantially microwave-transparent, has been incorporated into a microwave applicator and serves as the irradiation vessel. This reaction tube preferably has a diameter of one millimeter to approx. 50 cm, especially between 2 mm and 35 cm, for example between 5 mm and 15 cm. Reaction tubes are understood here to mean irradiation vessels whose ratio of length to diameter is greater than 5, preferably between 10 and 100 000, more preferably between 20 and 10 000, for example between 30 and 1000. In a specific embodiment, the reaction tube is configured in the form of a jacketed tube, through the interior and exterior of which the reaction mixture can be conducted successively in countercurrent, in order, for example, to increase the temperature control and energy efficiency of the process. The length of the reaction tube is understood to mean the total distance through which the reaction mixture flows. The reaction tube is surrounded over its length by at least one microwave radiator, but preferably by more than one microwave radiator, for example two, three, four, five, six, seven, eight or more microwave radiators. The microwaves are preferably injected through the tube jacket. In a further preferred embodiment, the microwaves are injected by means of an antenna via the tube ends.

The reaction tube is typically provided at the inlet with a metering pump and a manometer, and at the outlet with a pressure-retaining valve and a heat exchanger. The water-containing ammonium salt is preferably supplied to the reaction tube in liquid form at temperatures below 150° C., for example between 10° C. and 90° C. In a further preferred embodiment, amine and carboxylic acid, of which at least one component comprises water, are mixed only briefly before entry into the reaction tube. Additionally preferably, the reactants are supplied to the process according to the invention in liquid form with temperatures below 100° C., for example between 10° C. and 50° C. For this purpose, higher-melting reactants can be used, for example, in the molten state or admixed with solvent.

By varying tube cross section, length of the irradiation zone (this is understood to mean the proportion of the reaction tube within which the reaction mixture is exposed to microwave radiation), flow rate, geometry of the microwave radiators, the microwave power injected and the temperature attained, the reaction conditions are established such that the maximum reaction temperature is attained as rapidly as possible. In a preferred embodiment, the residence time at maximum temperature is selected to be sufficiently short that as low as possible a level of side reactions or further reactions occur. The continuous microwave reactor is preferably operated in monomode or quasi-monomode. The residence time in the reaction tube is generally less than 20 minutes, preferably between 0.01 second and 10 minutes, preferably between 0.1 second and 5 minutes, for example between one second and 3 minutes. To complete the reaction, the reaction mixture can pass through the reaction tube more than once, optionally after intermediate cooling.

In a particularly preferred embodiment, the aqueous ammonium salt is irradiated with microwaves in a reaction tube whose longitudinal axis is in the direction of propagation of the microwaves in a monomode microwave applicator. More particularly, the salt is irradiated with microwaves in a substantially microwave-transparent reaction tube which is present within a hollow conductor which is connected to a microwave generator and functions as a microwave applicator. The reaction tube is preferably aligned axially with a central axis of symmetry of this hollow conductor. The hollow conductor is preferably configured as a cavity resonator. Additionally preferably, the microwaves not absorbed in the hollow conductor are reflected at the end thereof. Configuration of the microwave applicator as a resonator of the reflection type achieves a local increase in the electrical field strength at the same power supplied by the generator, and increased energy exploitation.

The cavity resonator is preferably operated in E₀₁ₙ mode where n is an integer and states the number of field maxima of the microwave along the central axis of symmetry of the resonator. In this operation, the electrical field is directed in the direction of the central axis of symmetry of the cavity resonator. It has a maximum in the region of the central axis of symmetry and decreases to the value of zero toward the jacket. This field configuration is rotationally symmetric about the central axis of symmetry. According to the desired flow rate of the reaction mixture through the reaction tube, the required temperature and the required residence time in the resonator, the length of the resonator is selected relative to the wavelength of the microwave radiation used. n is preferably an integer from 1 to 200, more preferably from 2 to 100, particularly from 4 to 50, especially from 3 to 20, for example 3, 4, 5, 6, 7 or 8.

The microwave energy can be injected into the hollow conductor which functions as a microwave applicator through holes or slots of suitable dimensions. In a specific embodiment of the process according to the invention, the ammonium salt is irradiated with microwaves in a reaction tube present in a hollow conductor with a coaxial transition of the microwaves. Microwave devices particularly preferred for this process are constructed from a cavity resonator, a coupling device for injecting a microwave field into the cavity resonator and with one orifice each on two opposite end walls for passage of the reaction tube through the resonator. The microwaves are preferably injected into the cavity resonator by means of a coupling pin which projects into the cavity resonator. The coupling pin is preferably configured as a preferably metallic inner conductor tube which functions as a coupling antenna. In a particularly preferred embodiment, this coupling pin projects through one of the end orifices into the cavity resonator. The reaction tube more preferably adjoins the inner conductor tube of the coaxial transition, and is especially conducted through the cavity thereof into the cavity resonator. The reaction tube is preferably aligned axially with a central axis of symmetry of the cavity resonator, for which the cavity resonator preferably has one central orifice at each of two opposite end walls for passage of the reaction tube.

The microwaves can be fed into the coupling pin or into the inner conductor tube which functions as a coupling antenna, for example, by means of a coaxial connecting line. In a preferred embodiment, the microwave field is supplied to the resonator via a hollow conductor, in which case the end of the coupling pin which projects out of the cavity resonator is conducted into the hollow conductor into an orifice in the wall of the hollow conductor, and withdraws microwave energy from the hollow conductor and injects it into the resonator.

In a specific embodiment, the salt is irradiated with microwaves in a microwave-transparent reaction tube which is axially symmetric within an E₀₁ₙ round hollow conductor with a coaxial transition of the microwaves. In this case, the reaction tube is conducted through the cavity of an inner conductor tube which functions as a coupling antenna into the cavity resonator. In a further preferred embodiment, the salt is irradiated with microwaves in a microwave-transparent reaction tube which is conducted through an E₀₁ₙ cavity resonator with axial feeding of the microwaves, in which case the length of the cavity resonator is such that n=2 or more field maxima of the microwave develop. In a further preferred embodiment, the salt is irradiated with microwaves in a microwave-transparent reaction tube which is axially symmetric within a circular cylindrical E₀₁ₙ cavity resonator with a coaxial transition of the microwaves, in which case the length of the cavity resonator is such that n=2 or more field maxima of the microwave develop.

E₀₁ cavity resonators particularly suitable for the process according to the invention preferably have a diameter which corresponds to at least half the wavelength of the microwave radiation used. The diameter of the cavity resonator is preferably 1.0 to 10 times, more preferably 1.1 to 5 times and especially 2.1 to 2.6 times half the wavelength of the microwave radiation used. The E₀₁ cavity resonator preferably has a round cross section, which is also referred to as an E₀₁ round hollow conductor. It preferably has a cylindrical shape and especially a circular cylindrical shape.

The first advantage of the process according to the invention lies in an increased conversion of the reactants used compared to a reaction under comparable conditions without addition of water. For instance, the conversion is increased by addition of water typically by more than 1 mol %, in many cases by more than 5 mol %, in some cases by more than 10 mol %, for example by more than 20 mol %. This means that a lower level of reactants remains in the reaction mixture, which have to be removed and worked up or disposed of. In many cases, it has even been possible to obtain amides in directly marketable qualities by working in the presence of water in accordance with the invention. In addition, the handling specifically of low-boiling carboxylic acids and/or amines in the form of aqueous solutions is significantly simpler and more reliable than working with corresponding gases. Heat of neutralization released in the formation of the ammonium salt from carboxylic acid and amine is additionally at least partly absorbed by the water and can be removed more easily than from organic solvents. Furthermore, the presence of water as a solvent counteracts crystallization of the ammonium salts, such that costly and inconvenient heating of lines and vessels which contain reaction mixture before and after the microwave irradiation can be dispensed with.

### EXAMPLES

The microwave irradiation is effected in a single-mode microwave reactor of the “Initiator®” type from Biotage at a frequency of 2.45 GHz. The temperature was measured by means of an IR sensor. The reaction vessels used were closed, pressure-resistant glass cuvettes (pressure vials) with a volume of 5 ml, in which homogenization was effected by magnetic stirring. The temperature was measured by means of an IR sensor.

The microwave power was in each case adjusted over the experimental duration in such a way that the desired temperature of the reaction mixture was attained as rapidly as possible and then kept constant over the period specified in the experiment descriptions. After the microwave irradiation had ended, the glass cuvette was cooled with compressed air.

The reaction products were analyzed by means of ¹H NMR spectroscopy at 500 MHz in CDCl₃.

### Example 1

### Preparation of N,N-dimethyllactamide

A 500 ml three-neck flask with gas inlet tube, stirrer, internal thermometer and pressure equalizer was initially charged with 100 g of Lactol 90® (1 mol of lactic acid as 90% aqueous dilution). While cooling with ice, 45.1 g of gaseous dimethylamine (1 mol) were introduced slowly into the flask, and then the lactic acid N,N-dimethylammonium salt formed in a strongly exothermic reaction.

Aliquots were taken from this stock solution and adjusted by adding water to the water content specified in table 1.2 ml of each of these solutions were heated to a temperature of 225° C. in the microwave reactor, which established a pressure of about 20 bar. After attainment of thermal equilibrium (after approx. 1 minute), the mixture was kept at this temperature and this pressure with further microwave irradiation for two minutes. By means of ¹H NMR signal integration, the relative proportions of reactants and products in the reaction mixture were determined. The conversion rates are reproduced in the last column of table 1.

TABLE 1

| | Lactic acid N,N.- | water | Molar | Conversion to |
|---|---|---|---|---|
| | dimethyl- | [% by | ratio of | N,N-dimethyl- |
| Reaction | ammonium salt | wt.] | acid:amine | lactamide |
| (1) | 93% by wt. | 7 | 1:1 | 35 mol % |
| (2) | 64% by wt. | 36 | 1:1 | 48 mol % |
| (3) | 56% by wt. | 44 | 1:1 | 66 mol % |
| (4) | 47% by wt. | 53 | 1:1 | 90 mol % |
| (5) | 31% by wt. | 69 | 1:1 | 94 mol % |

### Example 2

### Preparation of N,N-dimethyl-4-methoxyphenylacetamide

A 500 ml three-neck flask with gas inlet tube, stirrer, internal thermometer and pressure equalizer was initially charged with 166.2 g of 4-methoxyphenylacetic acid (1 mol) which were neutralized gradually with 112.5 g of dimethylamine (as a 40% aqueous solution) while cooling. In a strongly exothermic reaction, the N,N-dimethylammonium salt of 4-methoxyphenylacetic acid formed. The solids content of the aqueous solution of this salt was 76%. A dilution of the salt to 50% was undertaken by adding further water to an aliquot of this solution.

In addition to the aqueous solutions, for comparison, the anhydrous ammonium salt was prepared and exposed to microwave radiation under the same conditions. To this end, a pressure vial was initially charged with 1.66 g of 4-methoxyphenylacetic acid with dry ice cooling, and then admixed rapidly with 0.45 g of condensed dimethylamine by means of a glass pipette precooled by dry ice. The vial was closed immediately and then thawed gradually, in the course of which the 4-methoxyphenylacetic acid N,N-dimethylammonium salt formed in an exothermic reaction. To homogenize the salt formation, the mixture was subsequently shaken vigorously and stirred with a magnetic stirrer bar.

2 ml of the ammonium salt or of the aqueous solutions thereof were in each case heated to a temperature of 235° C. in a microwave reactor, in the course of which a pressure of about 20 bar was established. On attainment of thermal equilibrium (after approx. 1 minute), the samples were held at this temperature and this pressure under further microwave irradiation for ten minutes. By means of ¹H NMR signal integration, the relative proportions of reactants and product in the reaction mixture were determined. The conversion rates achieved are reproduced in the last column of table 2.

TABLE 2

| | 4-Methoxyphenyl- | | | Conversion to |
|---|---|---|---|---|
| | acetic acid N,N- | Water | Molar | N,N-dimethyl- |
| React- | dimethyl- | [% by | ratio of | (4-methoxyphenyl)- |
| ion | ammonium salt | wt.] | acid:amine | acetamide |
| (6) | 100% by wt. | 0 | 1:1 | 8 mol % |
| (7) | 76% by wt. | 24 | 1:1 | 25 mol % |
| (8) | 50% by wt. | 50 | 1:1 | 41 mol % |

### Example 3

### Preparation of N,N.dimethyldecanamide

A 500 ml three-neck flask with gas inlet tube, stirrer, internal thermometer and pressure equalizer was initially charged with 172 g of decanoic acid (1 mol) which were cautiously neutralized with 112.5 g of dimethylamine (as a 40% aqueous solution). In an exothermic reaction, the decanoic acid N,N-dimethylammonium salt formed. The solids content of the pasty, aqueous formulation of the salt was 76% by weight. A dilution of the salt to 55% by weight was undertaken by adding further water to an aliquot of this solution.

In addition to the aqueous solutions, for comparison, the anhydrous ammonium salt was prepared and exposed to microwave radiation under the same conditions. A pressure vial was initially charged with 1.72 g of decanoic acid (0.01 mol) with dry ice cooling, and then admixed rapidly with 0.45 g of condensed dimethylamine (0.01 mol) by means of a glass pipette precooled by dry ice. The vial was immediately closed and then thawed cautiously with water cooling, which formed the decanoic acid N,N-dimethylammonium salt. To complete the salt formation, the mixture was shaken vigorously and stirred with a magnetic stirrer bar.

2 ml of the ammonium salt or of the aqueous solutions thereof were in each case heated to a temperature of 240° C. in the microwave reactor, which established a pressure of about 20 bar. On attainment of thermal equilibrium (after approx. 1 minute), the samples were kept at this temperature and this pressure under further microwave irradiation for ten minutes. By means of ¹H NMR signal integration, the relative proportions of reactants and product in the reaction mixture were determined. The conversion rates achieved are reproduced in the last column of table 3.

TABLE 3

| | Decanoic acid | Water | Molar | Conversion to |
|---|---|---|---|---|
| | N,N-dimethyl- | [% by | ratio of | N,N-dimethyl- |
| Reaction | ammonium salt | wt.] | acid:amine | decanamide |
| (9) | 100% by wt. | 0 | 1:1 | 15 mol % |
| (10) | 65% by wt. | 35 | 1:1 | 26 mol % |
| (11) | 49% by wt. | 51 | 1:1 | 35 mol % |

### Example 4

### Preparation of N,N-diethyl-m-toluamide

A 500 ml three-neck flask with gas inlet tube, stirrer, internal thermometer and pressure equalizer was initially charged with 136.2 g of m-toluic acid (1 mol) which were neutralized cautiously with 109.71 g of diethylamine (1.5 mol). In a strongly exothermic reaction, the m-toluic acid N,N-diethylammonium salt formed. Aliquots were taken from this stock solution and adjusted to the water contents specified in table 4 by adding water.

2 ml of the ammonium salt or of the aqueous solutions thereof were in each case heated to a temperature of 250° C. in the microwave reactor, which established a pressure of about 20 bar. On attainment of thermal equilibrium (after approx. 1 minute), the samples were kept at this temperature and this pressure under further microwave irradiation for 20 minutes. By means of ¹H NMR signal integration, the relative proportions of reactants and product in the reaction mixture were determined. The conversion rates achieved are reproduced in the last column of table 4.

TABLE 4

| | m-Toluic acid | Water | Molar | Conversion to |
|---|---|---|---|---|
| | N,N-dimethyl- | [% by | ratio of | N,N-dimethyl- |
| Reaction | ammonium salt | wt.] | acid:amine | decanamide |
| (12) | 100% by wt. | 0 | 1:1.5 | 5 mol % |
| (13) | 75% by wt. | 25 | 1:1.5 | 15 mol % |
| (14) | 65% by wt. | 35 | 1:1.5 | 19 mol % |
| (15) | 51% by wt. | 49 | 1:1.5 | 22 mol % |

1. A process for preparing a carboxamide comprising the steps of reacting at least one carboxylic acid of the formula I
R³—COON (I)
wherein R³ is hydrogen or a substituted or unsubstituted hydrocarbon radical having 1 to 50 carbon atoms with at least one amine of the formula II
HNR¹R² (II)
wherein R¹ and R² are each independently hydrogen or a substituted or unsubstituted hydrocarbon radical having 1 to 100 carbon atoms, or R¹ and R² together with the nitrogen atom to which they are bonded form a ring, forming an ammonium salt, and subsequently converting this ammonium salt to the carboxamide in the presence of superheated water under microwave irradiation, wherein water is added to the ammonium salt formed from carboxylic acid and amine before the irradiation with microwaves, and wherein the microwave irradiation is performed at temperatures above 150° C.

2. A process for preparing a carboxamide comprising the steps of reacting at least one carboxylic acid of the formula I
R³—COON (I)
wherein R³ is hydrogen or a substituted or unsubstituted hydrocarbon radical having 1 to 50 carbon atoms
with at least one amine of the formula II
HNR¹R² (II)
wherein R¹ and R² are each independently hydrogen or a substituted or unsubstituted hydrocarbon radical having 1 to 100 carbon atoms, or R¹ and R² together with the nitrogen atom to which they are bonded form a ring, in the presence of water to give an ammonium salt, and subsequently converting the ammonium salt thus prepared to the carboxamide at temperatures above 150° C. under microwave irradiation.

3. A process for increasing the conversion of microwave-supported amidation reactions, wherein water is added before microwave irradiation to an ammonium salt of at least one carboxylic acid of the formula I
R³—COOH (I)
wherein R³ is hydrogen or a substituted or unsubstituted hydrocarbon radical having 1 to 50 carbon atoms
and at least one amine of the formula II
HNR¹R² (II)
in which R¹ and R² are each independently hydrogen or a substituted or unsubstituted hydrocarbon radical having 1 to 100 carbon atoms, wherein the microwave irradiation is performed at temperatures above 150° C.

4. A process as claimed in claim 1, wherein the microwave irradiation is effected at pressures above atmospheric pressure.

5. A process as claimed in claim 1, wherein R³ is a hydrocarbon radical which has 1 to 50 carbon atoms and at least one substituent selected from the group consisting of C₁-C₅-alkoxy, poly(C₁-C₅-alkoxy), poly(C₁-C₅-alkoxy)alkyl, carboxyl, hydroxyl, ester, amide, cyano, nitrile, nitro, sulfo and aryl groups having 5 to 20 carbon atoms, where the C₅-C₂₀-aryl groups may have substituents selected from the group consisting of halogen atoms, halogenated alkyl radicals, C₁-C₂₀-alkyl, C₂-C₂₀-alkenyl, C₁-C₅-alkoxy, ester, amide, hydroxyl, hydroxyalkyl, cyano, nitrile and nitro groups.

6. A process as claimed in claim 1, wherein R³ is an aliphatic, cycloaliphatic, aromatic or araliphatic hydrocarbon radical.

7. A process as claimed in claim 1, wherein R³ comprises one or more double bonds.

8. A process as claimed in claim 1, wherein R¹ and R² are each independently a hydrocarbon radical having 1 to 100 carbon atoms.

9. A process as claimed in claim 1, wherein R¹ is a hydrocarbon radical having 1 to 100 carbon atoms and R² is hydrogen.

10. A process as claimed in claim 1, wherein R¹ or R² or both radicals are each independently, an aliphatic radical having 1 to 24 carbon atoms.

11. A process as claimed in claim 1, wherein R¹ and R² or both have substituents selected from the group consisting of hydroxyl, C₁-C₅-alkoxy, cyano, nitrile, nitro and C₅-C₂₀-aryl groups.

12. A process as claimed in claim 1, wherein R¹ or R² or both have C₅-C₂₀-aryl groups wherein the C₅-C₂₀-aryl groups have at least one substituent selected from the group consisting of halogen atoms, halogenated alkyl radicals, C₁-C₂₀-alkyl, C₂-C₂₀-alkenyl, C₁-C₅-alkoxy, ester, amide, cyano, nitrile and nitro groups.

13. A process as claimed in claim 1, wherein R¹ and R² together with the nitrogen atom to which they are bonded form a ring.

14. A process as claimed in claim 1, wherein R¹ and R² are each independently a radical of the formula III
—(R⁴—O)ₙ—R⁵ (III)
wherein
R⁴ is an alkylene group having 2 to 6 carbon atoms,
R⁵ is hydrogen or a hydrocarbon radical having 1 to 24 carbon atoms, and
n is from 2 to 50.

15. A process as claimed in claim 1, wherein R¹ and R² are each independently a radical of the formula IV
—[R⁶—N(R⁷)]ₘ—(R⁷) (IV)
wherein
R⁶ is an alkylene group having 2 to 6 carbon atoms or mixtures thereof,
each R⁷ is independently hydrogen, an alkyl or hydroxyalkyl radical having up to 24 carbon atoms, a polyoxyalkylene radical —(R⁴—O)ₚ—R⁵ or a polyimino-alkylene radical —[R⁶—N(R⁷)]_{q}—(R⁷), where R⁴, R⁵, R⁶ and R⁷ are each as defined above and q and p are each independently 1 to 50, and
m is from 1 to 20.

16. A process as claimed in claim 1, wherein the salt is irradiated with microwaves in a batchwise process.

17. A process as claimed in claim 1, wherein the salt is irradiated with microwaves in a continuous process.

18. A process as claimed in claim 17, wherein the salt is irradiated with microwaves in a substantially microwave-transparent reaction tube.

19. A process as claimed in claim 17, wherein the salt is irradiated with microwaves in a reaction tube whose longitudinal axis is in the direction of propagation of the microwaves of a monomode microwave applicator.

20. A process as claimed in claim 1, wherein the microwave irradiation is performed in the presence of 0.5 to 200% by weight of water based on the total mass of carboxylic acid and amine.

21. A process as claimed in claim 1, wherein the microwave irradiation is performed at temperatures above 180° C.

22. A process as claimed in claim 15, wherein m is from 2 to 10.
